(19) United States
(12) Patent Application Publication
(10) Pub. No.: US 2002/0104530 A1
(43) Pub. Date: Aug. 8, 2002

(54) Piezoelectric polymer flow sensor and methods

(75) Inventors: Yehuda Ivri, Irvine, CA (US); Niall Smith, The Crescent Galway (IE); John Power, Galway (IE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aerogen, Inc., Sunnyvale, CA

(21) Appl. No.: 10/005,454

(22) Filed: Nov. 2, 2001

Related U.S. Application Data
(63) Continuation-in-part of application No. 09/705,063, filed on Nov. 2, 2000.
Application No. 09/705,063 is a continuation-in-part of application No. 09/149,426, filed on Sep. 8, 1998, now Pat. No. 6,205,999.
Application No. 09/149,426 is a continuation of application No. 09/095,737, filed on Jun. 11, 1998, now Pat. No. 6,014,970.

(51) Int. Cl. A61M 11/00; B05B 17/06; A61M 15/00; A61M 16/10

(52) U.S. Cl. 128/200.16; 128/200.14; 128/203.12

(57) ABSTRACT

An aerosolization system comprises a gas flow passage, and an aerosol generator comprising a plate having a plurality of apertures and a vibratable element disposed to vibrate the plate. The aerosol generator is adapted to aerosolize a liquid for delivery into the gas flow passage. A flow sensor is configured to sense a gas flow through the passage, and a controller is employed to actuate the aerosol generator based on the gas flow sensed by the flow sensor.

### CROSS REFERENCE TO RELATED APPLICATIONS

This application is a continuation in part application and claims the benefit of U.S. application Ser. No. 09/705,063, filed Nov. 2, 2000, which is a continuation in part of U.S. application Ser. No. 09/149,426, filed Sep. 8, 1998 (now U.S. Pat. No. 6,205,999), which is a continuation of U.S. application Ser. No. 09/095,737, filed Jun. 11, 1998 (now U.S. Pat. No. 6,014,970), the complete disclosures of which are herein incorporated by reference.

### BACKGROUND OF THE INVENTION

This invention relates generally to the field of aerosolization, and in particular to the aerosolization of liquids, such as drug formulations. In one specific aspect, the invention relates to the automatic actuation of an aerosol generator upon user inhalation.

Oral drug delivery has many advantages over other routes of administration. For example, oral drug delivery does not require the formation of a separate access way into the body, such as by use of a needle, a transdermal patch, or the like. A variety of oral drug delivery techniques exist, including, for example, the use of pills, capsules, liquids, and aerosols, including both dry powder and liquid aerosols. Of particular interest to the invention are oral drug delivery techniques using aerosols where the user inhales from a mouthpiece to transfer the aerosolized drug into the lungs.

A variety of apparatus exists for aerosolizing medicaments. Merely by way of example, U.S. Pat. Nos. 5,140,740, 5,938,117, 5,586,550, 5,758,637, and 6,014,970, the complete disclosures of which are herein incorporated by reference, describe various aerosol generators that are capable of aerosolizing liquids using a vibratable aperture plate.

When inhaling an aerosolized medicament, it may be desirable to coordinate user inhalation with aerosol generation. For example, if the aerosol is generated after a user has inhaled a significant amount of air, the user may stop inhaling and start exhaling before the desired aerosol dose is delivered. On the other hand, if the drug is aerosolized too far in advance of user inhalation, some of the drug may not remain suspended and will therefore not be properly inhaled. In other cases, it may be desirable to coordinate operation of a ventilator with aerosol production so that an aerosol is not produced unless the ventilator is providing a gas stream to the user through the ventilator circuit for the user's inhalation phase of a breathing cycle.

Hence, this invention relates to devices and methods for coordinating drug aerosolization and gas flows, including those produced by user inhalation, ventilators and the like. In one aspect, the invention relates to the automatic generation of an atomized drug formulation upon user inhalation.

### SUMMARY OF THE INVENTION

The invention provides exemplary aerosolization devices and methods for aerosolizing liquids. In one embodiment, an aerosolization system comprises a gas flow passage and an aerosol generator for providing an aerosol into the gas flow passage. The aerosol generator comprises a plate having a plurality of apertures and a vibratable element disposed to vibrate the plate. A flow sensor is configured to sense a gas flow through the passage, and a controller is employed to actuate the aerosol generator based on the gas flow sensed by the flow sensor. In this way, aerosol production may be automated based on the gas flow sensed by the sensor.

Conveniently, the gas flow may be created by a user inhaling from a mouthpiece. With such a configuration, aerosol production does not begin until the user inhales. As another example, the gas flow passage may be coupled to a ventilator that produces inspiratory gas flow to a patient requiring assistance in breathing. In this manner, aerosol production may be configured to begin once the ventilator is actuated and the gas flow reaches the sensor.

In another particular embodiment, an aerosolization device comprises a housing having a mouthpiece, and an aerosol generator disposed in the housing. In one aspect, the aerosol generator may comprise a plate having a plurality of apertures and a vibratable element for vibrating the plate. In this way, operation of the aerosol generator aerosolizes a liquid that may be delivered to a user through the mouthpiece. The aerosolization device further includes a flow sensor to sense when a user inhales from the mouthpiece and to produce an electrical signal that is related to the rate of air flow created by the user when inhaling. A controller is employed to actuate the aerosol generator based on the electrical signal from the flow sensor. In this way, a user may simply inhale through the mouthpiece, with the controller actuating the aerosol generator once user inhalation is sensed. In this way, a drug will be aerosolized and ready for pulmonary delivery upon patient inhalation.

In one aspect, the controller of the invention may be configured to actuate the aerosol generator once the electric signal reaches a threshold value that is representative of an acceptable flow rate. In this way, the aerosol generator will not be actuated until the user produces a sufficient flow rate through the mouthpiece or a sufficient flow of air is flowing through a ventilator circuit to a user. If the flow rate produced by the user falls below the threshold rate, the controller may be configured to stop actuation of the aerosol generator to ensure that a drug will only be aerosolized when the user produces a sufficient flow rate.

In one particular aspect, the sensor of the invention may be constructed of a flexible member having material with an electrical resistance that changes upon bending of the flexible member. For example, the electrical signal may comprise a voltage change that is proportional to the amount of bending of the flexible member. Conveniently, the controller may include circuitry to detect voltage changes and to actuate the aerosol generator when a threshold voltage change is detected. In one specific aspect, the flexible member may comprise a thin sheet having a layer of strain sensitive polymer that experiences a change of electrical resistance when the thin sheet bends. Conveniently, the flexible member may be disposed within a flow path that extends through the housing or through the ventilator circuit so that the flexible member will bend upon patient inhalation.

As another example, the sensor may comprise a bendable member that produces a voltage change upon bending that is related to the rate of gas flow created by the user when inhaling. In one embodiment, the bendable member may comprise a piezoelectric polymer that produces a voltage depending on the stress applied during bending. With such a sensor, a charge amplifier may be used to detect the voltage change produced by the bendable member and then to send this signal to the controller. The output from the charge amplifier may be further amplified until the desired sensitivity to flow is obtained. The piezoelectric polymer may be constructed as a thin film flap that is placed in the airway of the housing or within the ventilator circuit, to be bent by inspiratory air flow. The sensor may be sufficiently flexible so that it has essentially no significant resistance to flow.

In another particular aspect, the controller may be configured to measure the volume of air passing through the mouthpiece or through the ventilator circuit to the user based upon the voltage change over time. In this way, the user's tidal volume may be calculated. Further, the controller may be used to determine the amount of time during which the aerosol generator is operated to determine whether a unit dosage has been aerosolized.

The invention further provides an exemplary method for aerosolizing a liquid using such an aerosolization device. According to the method, the user inhales from the mouthpiece to produce a flow of air through the mouthpiece. The flow rate of the air passing through the mouthpiece is sensed with the sensor, and an electrical signal that is related to the flow rate is transmitted to the controller. A signal is then sent from the controller to vibrate the vibratable element when the flow rate reaches a threshold rate. Conveniently, operation of the aerosol generator may be stopped when the flow rate falls below a threshold rate.

In one exemplary step, the flow sensor comprises a flexible member having a material with an electrical resistance that changes upon bending of the flexible member. In this way, the controller may be employed to detect a voltage change that is proportional to the flow rate. Once the threshold flow rate has been reached, an electrical signal may be sent from the controller to a piezoelectric transducer to vibrate the plate and aerosolize the liquid. Alternatively, a piezoelectric flap may be used to produce a voltage change when bent by the air flowing through the housing or through the ventilator circuit for inhalation by a user.

In another step, the controller may be employed to calculate the volume of air passing through the mouthpiece based on the flow rate over time. This may then be used to determine the tidal volume of user.

### BRIEF DESCRIPTION OF THE DRAWINGS

FIG. 1 is a cross sectional schematic diagram of an aerosolization device having a flow sensor according to the invention.

FIG. 2 illustrates the aerosolization device of FIG. 1 when air is flowing through the device to actuate the flow sensor.

FIG. 3 is a perspective view of one embodiment of a flow sensor according to the invention.

FIG. 4 is a flow chart illustrating one method for aerosolizing a liquid upon user inhalation according to the invention.

FIG. 5 is a schematic diagram of an alternative flow sensor according to the invention.

### DESCRIPTION OF THE SPECIFIC EMBODIMENTS

In one embodiment, the invention provides exemplary aerosolization devices and techniques which permit the synchronization of aerosolization with user inhalation. Synchronization is accomplished by sensing the flow of air through the aerosolization device when the user inhales and actuating the aerosolization device when the air flow is sensed. Hence, the techniques of the invention may be used with essentially any aerosolizer where the patient inhales through a mouthpiece or through a ventilator circuit to deliver the drug to the lungs. Merely by way of example, the invention may be used with atomizers which atomize a liquid medicament, such as those described in U.S. Pat. Nos. 5,140,740, 5,938,117, 5,586,550, 5,758,637, and 6,014,970, previously incorporated by reference. However, it will be appreciated that the invention is not intended to be limited to only these specific atomizers, but may be used with any atomization device where an air flow is provided through the device as just described. For example, the invention may also be used with essentially any type of ventilator, with the sensor being used to sense when the ventilator is providing a predetermined air flow to a ventilated patient. As another example, the sensor may be used to detect when a gas flow from the ventilator has reached the vicinity of the aerosol generator. Once the gas flow is sensed, the aerosol generator may be actuated. In this way, aerosol production is not tied directly to actuation of the ventilator unit. As a further example, the sensor may be used when the ventilator is placed in an assist mode to sense when a user inhales. This signal is used to actuate the ventilator as well as to actuate the aerosol generator. Merely by way of example, one type of ventilator that may be used with the invention is described in copending U.S. application Ser. No. 09/849,194, filed May 4, 2001, the complete disclosure of which is herein incorporated by reference.

Conveniently, the aerosolization devices may be configured to have a trigger so that aerosolization will only occur once a threshold flow rate through the device has been met or exceeded. In another option, the device may be configured to stop aerosolization in the event that the flow rate falls below the threshold value. In this way, atomization will automatically occur as long as the user produces an acceptable flow rate that is sufficient to deliver the aerosolized medicament to the patient's lungs.

Another feature is the ability to calculate the volume of air flowing through or past the aerosolization device using the sensor. This is accomplished by measuring the flow rate of air over time. In this way, the aerosolization device may be employed to measure the user's tidal volume. Further, the aerosolization devices may be configured to keep a record of actuation times to ensure that a complete dosage has been supplied to the user.

In one embodiment, the flow sensor is configured to produce an electrical signal that is related to the flow rate. This may be accomplished, for example, by providing a flexible member with a material whose resistance changes as the flexible member bends due to the air flow created by the user. The change in resistance produces a voltage drop that is related to the flow rate. A controller may then be employed to trigger operation of the device when a threshold voltage has been sensed. A variety of materials may be employed to produce the change of voltage based on the deflection of the flexible member, including, for example, resistive inks, strain sensitive polymers, and the like. Examples of materials that may be used are also described in U.S. Pat. Nos. 5,086,785, 5,157,372 and 5,309,135, the complete disclosures of which are herein incorporated by reference. As another example, a voltage change may be produced by bending a piezoelectric polymer flap. The flap is placed across the air flow conduit and bends as the user inhales. The voltage change may be detected by a charge amplifier and then sent to the controller to control operation of the aerosol generator.

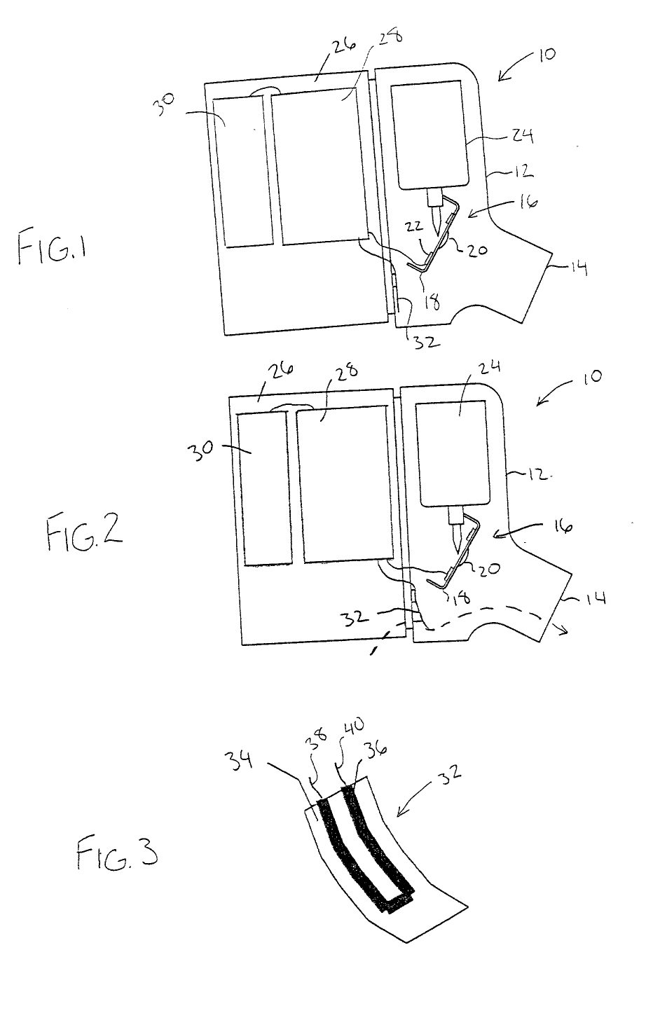

Referring now to FIG. 1, one embodiment of an aerosolization device 10 will be described. Device 10 comprises a housing to hold the various components of aerosolization device 10. Housing 12 further includes a mouthpiece 14 and one or more vents (not shown) to permit air to enter into housing 12 when a user inhales from mouthpiece 14. Disposed within housing 12 is an aerosol generator 16 that comprises a cup shaped member 18 to which is coupled an aperture plate 20. An annular piezoelectric element 22 is in contact with aperture plate 20 to cause aperture plate 20 to vibrate when electrical current is supplied to piezoelectric element 22. Aperture plate 20 is dome shaped in geometry and includes a plurality of tapered apertures that narrow from the rear surface to the front surface. Exemplary aperture plates and aerosol generators that may be used in aerosolization device 10 are described in U.S. Pat. Nos. 5,140,740, 5,938,117, 5,586,550, 5,758,637, and 6,014,970, previously incorporated by reference.

Aerosolization device 10 further includes a canister 24 having a supply of liquid that is to be aerosolized by aerosol generator 16. Canister 24 may include a metering valve to place a metered amount of liquid onto aperture plate 20. Although not shown, a button, electrical solenoid, or the like may be employed to dispense the volume of liquid when requested by the user.

Housing 12 includes an electronics region 26 for holding the various electrical components of aerosolization device 10. For example, region 26 may include a printed circuit board 28 which serves as a controller to control operation of the aerosol generator 16. More specifically, circuit board 28 may send an electrical signal to piezoelectric element 22 to vibrate aperture plate 20. A power supply 30, such as one or more batteries, is electrically coupled to circuit board 28 to provide aerosolization device 10 with power.

Also disposed within housing 12 is a flow sensor 32 that is electrically coupled to circuit board 28. Flow sensor 32 is positioned across a flow path extending between one or more inlet vents and mouthpiece 14. Flow sensor 32 is configured as a bend sensor that bends when a user inhales from mouthpiece 14 to create a flow of air through housing 12 as shown in FIG. 2. Flow sensor 32 bends in proportion to the rate of air flowing through housing 12. Flow sensor 32 is also configured to produce an electrical signal that is proportional to the amount of bending. The signal is transferred to circuit board 28 which may be configured to send an electrical signal to piezoelectric element 22 when a threshold voltage drop has been produced. As long as the threshold voltage drop is maintained, current will be supplied to aerosol generator 16. If, however, the user produces a flow rate which causes the voltage drop to fall below the threshold value, circuit board 28 will stop supplying electrical current to aerosol generator 16, thereby stopping the aerosolization process. Further, circuit board 28 may be configured to record the flow rate of air over time to measure the volume of air flowing through mouthpiece 14. In this way, circuit board 28 may be employed to determine the user's tidal volume.

Hence, aerosolization device 10 permits a user to coordinate actuation of aerosol generator 16 with their own inhalation simply by inhaling at a sufficient flow rate. This causes flow sensor 30 to bend sufficient to create the necessary voltage drop to permit circuit board 28 to actuate aerosol generator 16. When the user stops inhaling, aerosol production is also stopped. Conveniently, circuit board 28 may also be configured to measure the time during which aerosol generator 16 is actuated to ensure that the user has inhaled for a sufficient time to aerosolize all of the drug supplied from canister 24.

Referring now to FIG. 3, flow sensor 32 will be described in greater detail. Flow sensor 32 comprises a thin flexible sheet 34 having a thin layer of strain sensitive polymer 36 that is disposed on sheet 34. A pair of electrical leads 38 and 40 are electrically coupled to polymer 36 to measure a change of voltage that is proportional to the amount of bending of sheet 34. Hence, circuit board 28 (see FIG. 1) may be electrically coupled to leads 38 and 40 and include circuitry to measure a voltage drop as the user inhales from mouthpiece 14. This information may then be employed to measure the flow rate through mouthpiece 14. In one particular embodiment, flexible sheet 34 may have width of about 5 mm, a length of about 20 mm, and a thickness of about 20 microns. Examples of sensors that may be used in aerosolization device include sensors commercially available from Flexpoint Flexible Sensor Systems of Midvale, Utah, and may be designated a Bend Sensor®.

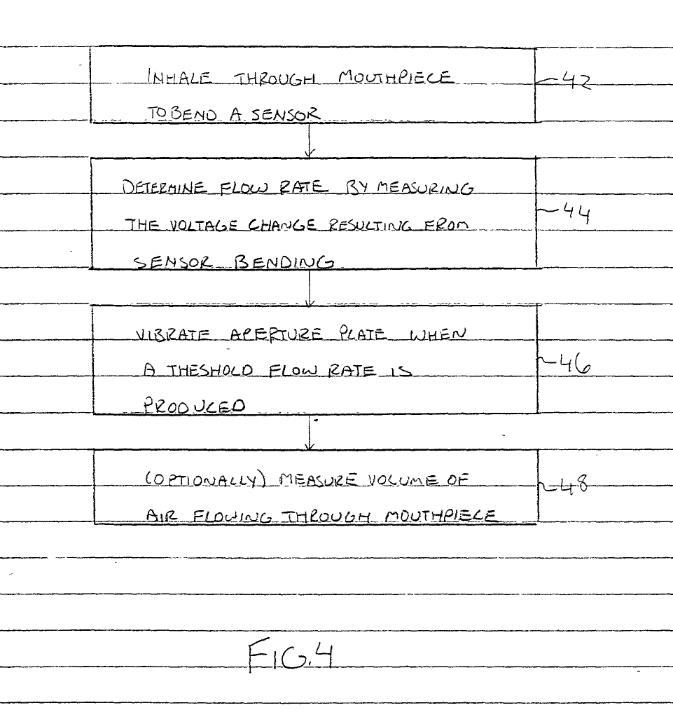

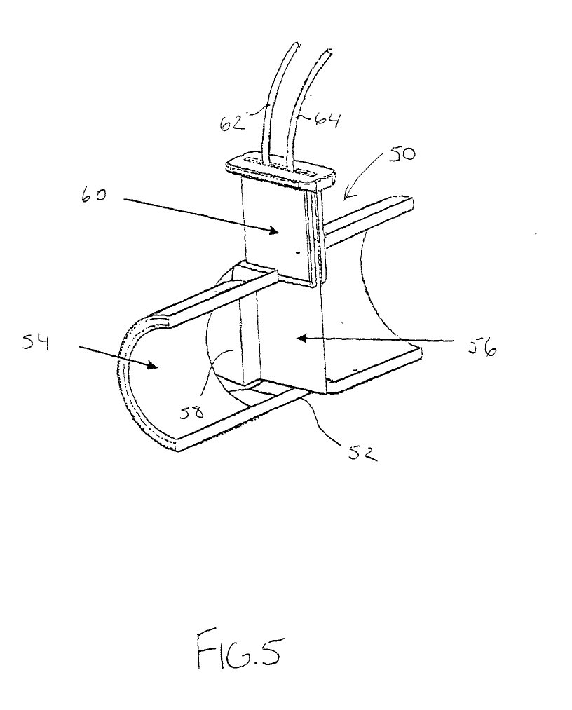

Referring now to FIG. 4, one method for aerosolizing a liquid based on an acceptable flow rate produced by a user will be described. The process begins at step 42 where the user inhales through a mouthpiece of an aerosolization device to bend a flow sensor. The aerosolization device may be a hand held aerosolizer or may be incorporated with a ventilator circuit of a ventilator to provide aerosol to a ventilated patient or the like. The flow rate of air through the device is determined by measuring the voltage change resulting from bending of the sensor as shown in step 44. Once a threshold flow rate is produced, a signal is sent to vibrate the aperture plate to aerosolize a volume of liquid previously supplied to the aperture plate as shown in step 46. As long as the threshold flow rate is met or exceeded, the aperture plate is vibrated. Optionally, the volume of air flowing through the mouthpiece may be determined by integrating the flow rate over time as shown in step 48. Also, the time during which the aperture plate is vibrated may be tracked to ensure that all of the liquid has been aerosolized and inhaled by the user.

FIG. 5 illustrates another embodiment of a flow sensor 50. As shown, flow sensor 50 is included within a ventilator tube 52. However, it will be appreciated that flow sensor may be used within essentially any type of aerosolization device as previously described. Conveniently, tube 52 may include a connector 54 to facilitate coupling of tube 52 to the rest of the ventilator.

Flow sensor 50 comprises a piezoelectric polymer flap 56 that is disposed across tube 52. Flap 56 bends depending on the air flow passing through tube 52. A stop 58 is provided within tube 52 to prevent bending of flap 56 when air is blown into tube 52. Flap 56 is constructed of a thin polymer piezoelectric film that produces a voltage change when stressed, such as when bent. Flap 56 is sufficiently flexible to that is provides essentially no significant resistance to flow as the user inhales, i.e. flap 56 easily bends to permit air flow through tube 52. Examples of such a material is a 50 μm piezoelectric film, commercially available from Measurement Specialists Inc., Sensor Division, Norristown, Pa. In this way, flap 56 may be constructed to be relatively inexpensive while also having good sensitivity. For example, flap 56 may detect flows as low as 5 Lpm.

Flap 56 includes electrodes (hidden from view) that permit flap 56 to be electrically coupled to a connector 60. Conveniently, the electrodes may be constructed of a Cu/Ni alloy. The electrodes are coupled to wires 62 and 64 of connector 60 that permit flap 56 to be electronically coupled to other circuitry, such as a controller for controlling the aerosol generator. The electrodes may be attached to wires 62 and 64 using an adhesive copper strip to which wires 62 and 64 have been soldered. Alternatively, a crimping approach may be used.

Flap 56 produces a voltage depending on the stress applied. A charge amplifier (not shown) may be coupled to wires 62 and 64 to detect movement of flap 56. The charge amplifier has a leak resistance to ensure the output voltage does not drift up to the supply rails. This output is further amplified until the required sensitivity to flow is obtained. The output signal may be used by a controller to control operation of the ventilator similar to other embodiments. Because the output is generally linear with flow, sensor 50 may also be used as a volumetric flow sensor in a manner similar to that described with other embodiments.

Sensor 50 may be placed either upstream or downstream of an aerosol generator. For example, in cases where tube 52 is coupled to a ventilator unit, sensor 50 may be disposed just upstream of an aerosol generator that is configured to eject an aerosol into tube 54. Such an aerosol generator may be similar to any of those described herein. The aerosol generator controller may be configured to receive electrical signals from wires 62 and 64 and to send a signal to start aerosol production once a gas flow has been detected within tube 52. In this way, a user may simply actuate the ventilator, and aerosol production will automatically begin once the gas flow produced by the ventilator reaches flap 56. Once the ventilator is turned off, gas flow will stop and will cause operation of the aerosol generator to also stop. Alternatively, sensor 50 may be used when the ventilator unit is in a breathing assist mode to actuate the ventilator after sensing when the user breathes through the ventilator tube. Once the gases from the ventilator unit reach sensor 50, another signal may be sent to actuate the aerosol generator.

The invention has now been described in detail for purposes of clarity of understanding. However, it would be appreciated that certain changes and modifications may be practiced within the scope of the appended claims.

What is claimed is:

1. An aerosolization system comprising:
a gas flow passage;
an aerosol generator comprising a plate having a plurality of apertures and a vibratable element disposed to vibrate the plate, wherein the aerosol generator is adapted to aerosolize a liquid for delivery into the gas flow passage;
a flow sensor that is configured to sense a gas flow through the passage; and
a controller to actuate the aerosol generator based on the gas flow sensed by the flow sensor.

2. A system as in claim 1, wherein the sensor comprises a bendable member that produces a voltage change upon bending that is related to the rate of gas flow through the passage, and wherein the controller is configured to actuate the aerosol generator when the voltage change reaches a threshold value that is representative of an acceptable flow rate.

3. A system as in claim 2, wherein the bendable member comprises a piezoelectric polymer, and wherein the sensor further comprises a charge amplifier to detect the voltage change produced by the bendable member.

4. A system as in claim 3, wherein the controller includes circuitry to receive a signal from the charge amplifier and to actuate the aerosol generator when a threshold voltage change is detected.

5. A system as in claim 3, wherein the bendable member further comprises a thin sheet fashioned as a flap.

6. A system as in claim 5, wherein the flap is disposed at least partially across the passage.

7. A system as in claim 3, wherein the controller includes circuitry to stop operation of the aerosol generator when the voltage change falls below the threshold voltage.

8. A system as in claim 3, wherein the controller further includes circuitry to determine the volume of air passing through the mouthpiece based on the voltage change over time.

9. A system as in claim 1, wherein the vibratable element comprises a piezoelectric transducer, and wherein the controller further includes circuitry to send an electrical signal to the piezoelectric transducer to vibrate the plate.

10. A system as in claim 1, further comprising a housing having a mouthpiece, wherein the aerosol generator is disposed in the housing, and wherein the passage leads to the mouthpiece such that inhaling from the mouthpiece creates the gas flow.

11. A system as in claim 1, further comprising a ventilator and a tube extending from the ventilator, wherein the passage is defined by the tube, and wherein the ventilator is configured to produce the gas flow.

12. A method for aerosolizing a liquid, the method comprising:
providing an aerosol generator, a gas flow passage, a flow sensor, and a controller, wherein the aerosol generator comprises a plate having a plurality of apertures and a vibratable element disposed to vibrate the plate to dispense an aerosol into the gas flow passage;
creating a has flow through the passage;
sensing the gas flow through the passage with the sensor and transmitting an electrical signal to the controller;
sending a signal from the controller to vibrate the vibratable element upon receipt of the electrical signal.

13. A method as in claim 12, wherein the flow sensor comprises a bendable member that produces a voltage change upon bending that is related to the rate of gas flow through the passage, wherein the sensor senses the flow rate of the gas through the passage and sends the signal to the controller to vibrate the vibratable element when the flow rate reaches a threshold value.

14. A method as in claim 13, further comprising stopping operation of the aerosol generator if the flow rate falls below the threshold rate.

15. A method as in claim 13, further comprising calculating with the controller the volume of air passing through the mouthpiece based on the flow rate over time.

16. A method as in claim 13, wherein the bendable member comprises a piezoelectric polymer, and wherein the sensor further comprises a charge amplifier to detect the voltage change produced by the bendable member.

17. A method as in claim 12, wherein the vibratable element comprises a piezoelectric transducer, and further comprising sending an electrical signal from the controller to the piezoelectric transducer to vibrate the plate.

18. A method as in claim 12, wherein the aerosol generator is disposed in a housing having a mouthpiece that is connected to the passage, and further comprising inhaling from the mouthpiece to create the gas flow.

19. A method as in claim 12, further comprising a ventilator and a tube extending from the ventilator, wherein the passage is defined by the tube, and operating the ventilator to produce the gas flow.
